(19) United States
(12) Patent Application Publication
(10) Pub. No.: US 2007/0179446 A1
(43) Pub. Date: Aug. 2, 2007

(54) Device with a slidable safety member for positioning a cannula in a vein

(75) Inventors: Jean-Luc Carrez, Ecouen (FR); Pierrick Guyomarc'h, Ermont (FR); Valery Dalle, Gouvieux (FR); Jean-Max Huet, Clichy (FR)

Correspondence Address: LEVINE & MANDELBAUM, 444 MADISON AVENUE, 35TH FLOOR, NEW YORK, NY, 10022, US

(73) Assignee: Vygon, Ecouen (FR)

(21) Appl. No.: 10/598,432

(22) PCT Filed: Mar. 2, 2005
(86) PCT No.: PCT/FR05/00493

§ 371 Date: Aug. 30, 2006

(30) Foreign Application Priority Data
Mar. 2, 2004 (FR) 0402121

(51) Int. Cl. A61M 5/178; A61M 31/00

(52) U.S. Cl. 604/164.01; 604/508; 604/164.08

(57) ABSTRACT

The invention relates to positioning a cannula in a vein. A tab (11) is provided on a movable member (9, 10) comprising a wall (10) with a through-hole (12) therein for inserting a needle in such a way that inserting the needle into the through-hole holds the tab in a retaining position on a flange of the base (2) of a catheter (1), wherein said wall (10) is transversely slidably mounted in the chamber of the needle stick guard (6) relative to the needle between a lower retaining position and an upper release position so that when the needle is retracted into the guard and spaced back from said hole, the wall (10) that no longer has the needle extending therethrough can slide into the upper position thereof, thereby raising said tab above the flange of the base and enabling the guard to be separated from the catheter base. The invention is suitable for use in intravenous catheters.

This present invention concerns devices used for the insertion into a vein of a cannula composed of a short tubular catheter with a proximal base, by means of a puncture needle.

The insertion procedure includes a puncture stage in which the needle is pushed into the catheter base and into the catheter so that its point exits at the distal end of the catheter and in which, with this point, the operator punctures the vein into which he wishes to insert the catheter, together with an insertion stage in which the operator slides the catheter along the needle in the distal direction causing the catheter to enter into the vein, and a withdrawal stage in which the operator withdraws the needle from the vein, from the catheter and from the catheter base.

At the end of the withdrawal stage, the point of the needle is exposed to the open air and there is a risk that the operator who is holding the catheter and its base with one hand, and who is holding the needle with the other hand, has inadequate control over the needle and can pierce his skin with its point.

Publication EP 0832 666 describes a arrangement which includes an intermediate part between the base of a cannula and the end of a tube which is used to trap the needle.

The intermediate part is designed at one end for fitting the part on the base of the cannula, and at its opposite end for the fitting onto the part of the tube intended to trap the needle.

The intermediate part is not intended to trap the needle, but to provide a sealing gasket.

The fitting of the base of the cannula onto this part is effected by screwing or by conical trapping, which does not prevent the part from being detached from this base accidentally before the needle is withdrawn.

Though this arrangement has a temporary means of attachment of the tube which surrounds the needle onto the intermediate part, this attachment does not concern the temporary attachment of the intermediate part onto the base of the cannula.

Publication U.S. Pat. No. 4,900,307 describes an arrangement which is designed only for withdrawing the needle into a tube at the end of which a nose is fixed. After the fitting of the needle in the arrangement, this nose is fixed permanently to the end of the tube. The arrangement includes a trigger that can be operated manually in order to trigger the removal of the needle.

Also known is the temporary attachment, in the extension toward the rear of the catheter base, a detachable cage through which the needle can slide and which is equipped with a trap to hold the puncture end of the needle in the chamber when the latter exits from the base and to remain in position on this end when the cage is detached from the base.

In order to attach the cage temporarily to the base of the needle, a known arrangement creates friction-type conical interlocking of the cage in or on the base of the needle, so that the cage detaches from the base under the effect of traction applied axially to the needle after the puncture end of the needle has entered into the cage (EP 0 456 694 or U.S. Pat. No. 5,322,517, U.S. Pat. No. 5,135,504, U.S. Pat. No. 5,176,655, and others).

The risk still exists however if the cage detaches from the base prematurely before the puncture end of the needle is trapped in the cage.

In order to remove this risk, it has been recommended that the cage be fitted with a transversally mobile device, held by the needle in a position in which it is maintained by the catheter base, and capable of returning of its own accord to a position of freedom when the puncture end of the needle is withdrawn into the cage.

Publication EP 0 891 198 or U.S. Pat. No. 6,001,080 achieves this retention by the penetration, into a cavity formed on the inner face of the wall of the catheter base, of a dog formed on a wall of the cage, the said dog being held in a retention position by a lateral contact with the needle and being released and capable of moving radially to escape from the cavity when this lateral contact is eliminated by removal of the needle behind the dog.

This retention arrangement, which is totally concealed in the base and in the cage, is difficult to control, and the automatic radial movement of the lug can be insufficient to free the cage from the base.

Publication U.S. Pat. No. 6,234,999 describes another retention arrangement in which the cage has a device fitted with a dog which is held by an external collar of the base but which is not held by the needle, so that an unintended traction on the cage can end this retention prematurely.

This present invention has as its objective to provide a cage equipped with a retention arrangement to overcome the aforementioned drawbacks, and in particular a retention arrangement which is both visible at the outside of the cage and the base, and held by the needle in a retention position for as long as the puncture end of the needle is not withdrawn into the cage.

One subject of the invention is therefore an arrangement for the insertion, into a vein, of a cannula composed of a short tubular catheter with a proximal base, where this arrangement includes a needle with a puncture end and a cage which extends the base of the cannula in the proximal direction, where this chamber forms a chamber through which the needle slides from a proximal entrance to a distal exit, and that is equipped with a trap to hold the puncture end of the needle in the chamber when the needle is withdrawn from the catheter base, with the cage and the base being equipped with retention resources that combine so as to perform temporary retention of the cage on the base before the puncture end of the needle is trapped in the chamber of the cage, with the said retention resources including an external rim formed on the base and an external dog provided on the cage to be held by this rim, characterised in that the dog is formed on a mobile device which includes a wall traversed by a hole for the passage of the needle so that the passage of the needle in the hole holds the dog in a retention position, the said wall being mounted to slide in the chamber of the cage in a direction transverse to the needle between a down held position and an up released position so that when the needle is withdrawn into the cage until it has moved to the rear of the said hole, the wall which is not traversed by the needle can slide to its up position, lifting the said dog and allowing the cage to separate from the catheter base.

In particular implementations, the arrangement of the invention has one or more of the following characteristics:
the dog is formed at the end of a wall of the safety device which is at right angles to the sliding wall;
the collar and/or the dog has a ramp so that a reverse movement of the cage in the proximal direction causes the dog to lift when the needle no longer traverses the said hole;
resources are provided to limit the lifting of the sliding wall in the up position;
resources are provided to prevent the mobile device returning to the down position after it has reached the up position;
the cage is formed of a rear part which constitutes the chamber and the entrance of the chamber, and of a front part which includes a transverse wall which closes off the said chamber and which includes the exit of the chamber, with this transverse wall forming a slide for the said sliding wall of the said mobile device, these two parts being held together by the click-on action of nipples provided on the sides of the transverse wall of the front part, in holes formed in the wall of the chamber of the rear part, or vice versa;
ahead of the said transverse wall, the front part of the cage constitutes a nose designed to fit into in the catheter base;
the transverse wall of the front part of the cage includes a nipple which slides in an oblong aperture of the sliding wall of the mobile device in order to limit the lifting of the mobile device;
the sliding wall of the mobile device has flexible and elastic lateral legs which are constrained by rims formed on the transverse wall of the front part of the cage for as long as the device has not yet reached the up position, and which deploy when the mobile device reaches the up position, to rest on these rims and so prevent the mobile device from returning to the down position. The rear part of the cage is shaped so that the base of the needle slots into this part for the puncture operation;
resources are provided to prevent the point of the needle from exiting via the entrance of the chamber.

A description follows of one non-limiting implementation as an example of the scope of the invention, with reference the appended figures in which:

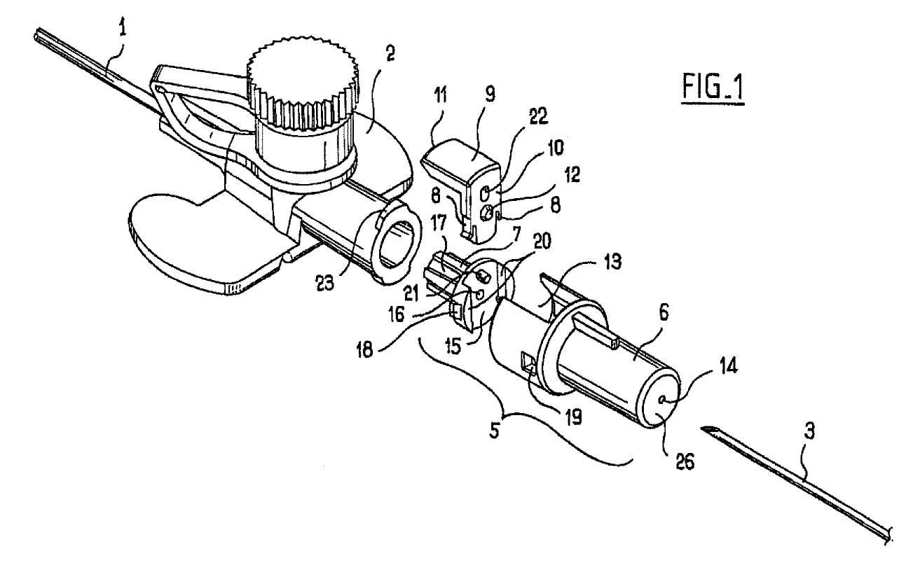

FIG. 1 is a view in perspective of the arrangement in which the short catheter with its base, the needle, the two parts of the cage and the sliding retention device have been shown separately;

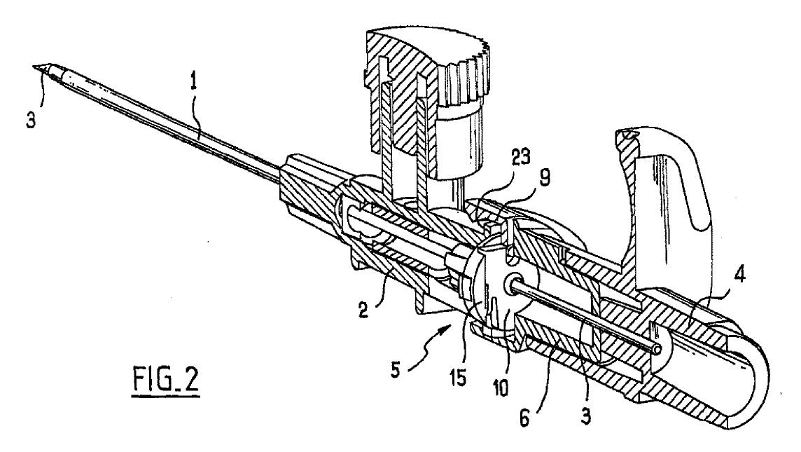

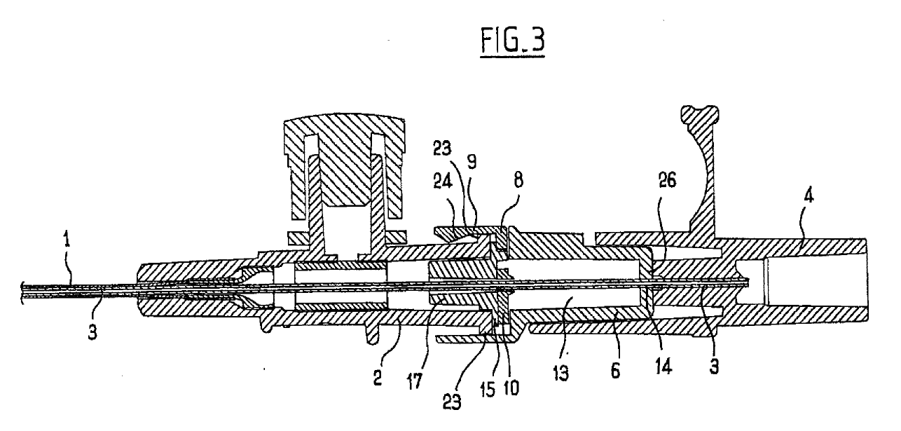

FIGS. 2 and 3 represent the arrangement ready for use, in stripped perspective and in axial section respectively;

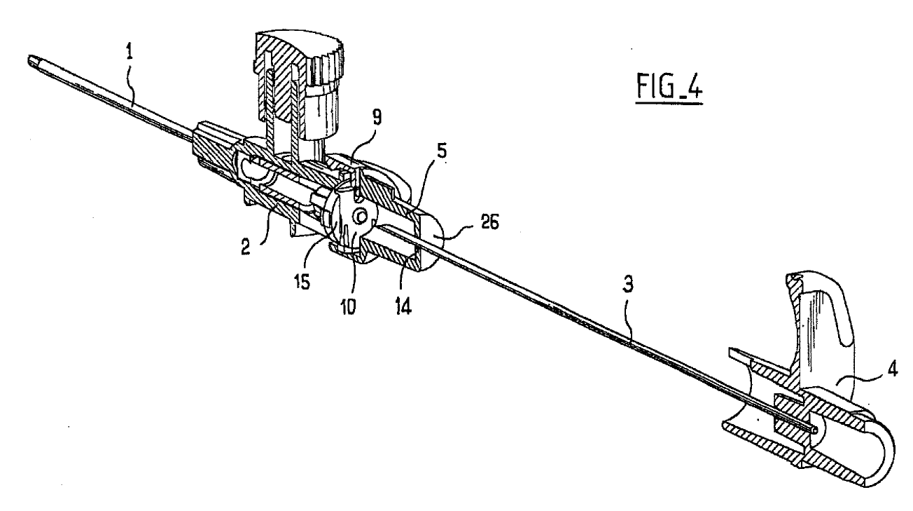

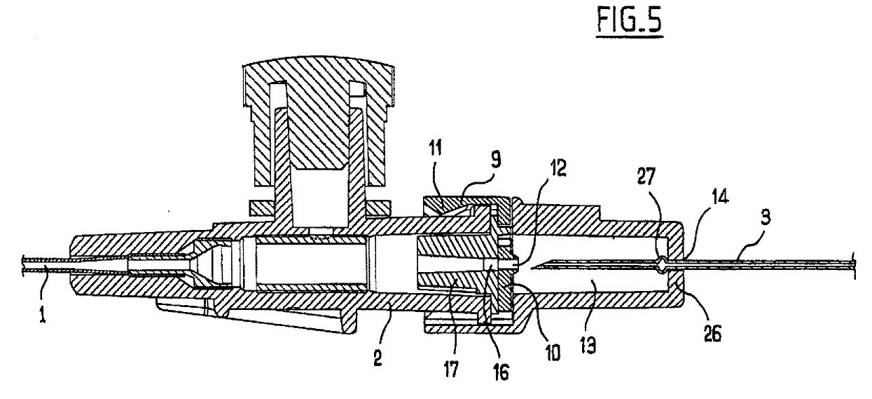

FIGS. 4 and 5 represent the arrangement, respectively in stripped perspective and in axial section respectively, with the point of the needle withdrawn into the chamber of the cage;

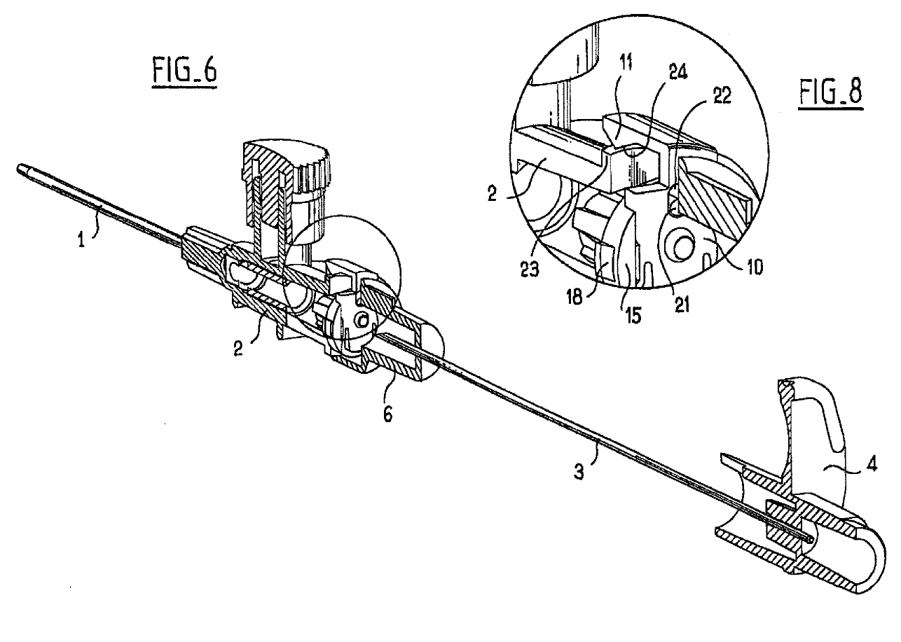

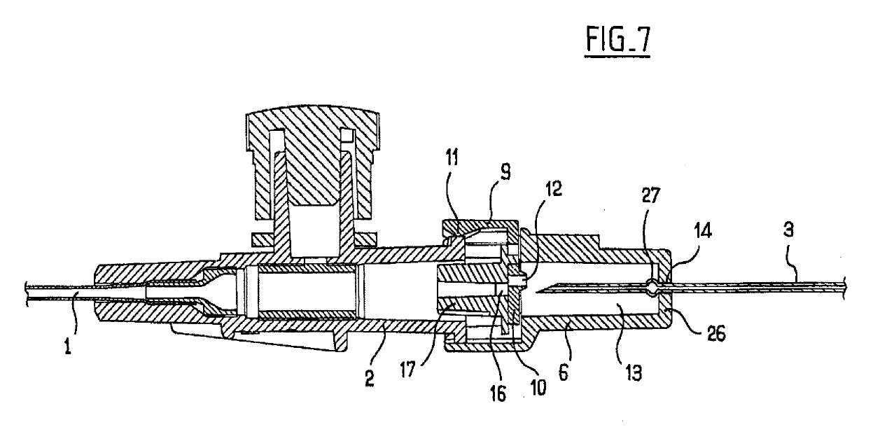

FIGS. 6 and 7 represent the arrangement, in stripped perspective and in axial section respectively, while the retention device is in the process of lifting.

FIG. 8 is a magnified view of a detail of FIG. 6;

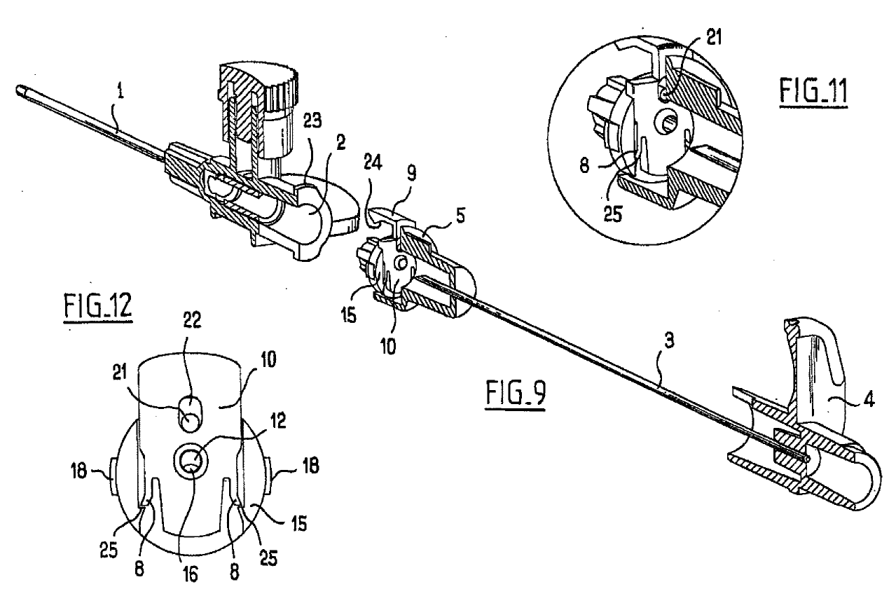

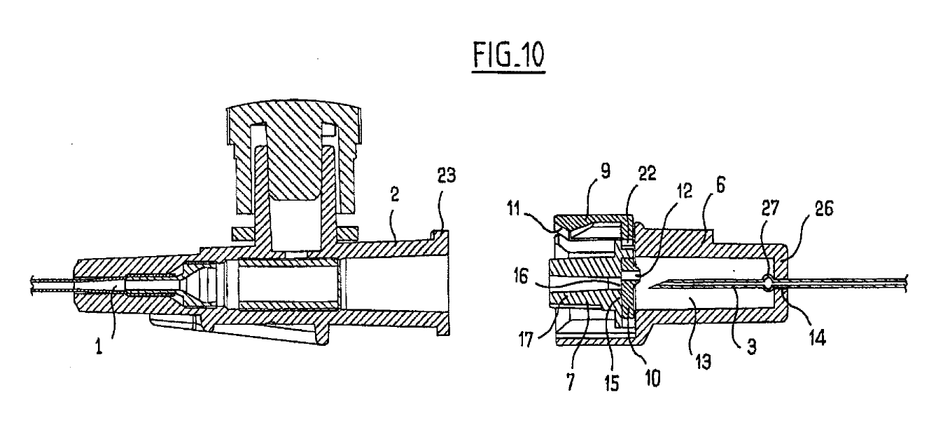

FIGS. 9 and 10 represent the arrangement, in stripped perspective and in axial section respectively, with the cage detached from the catheter base;

FIG. 11 is a magnified view of a detail of FIG. 9, and

FIG. 12 is a plan view of the transverse wall of the front part, partially masked by the sliding wall of the retention device.

The arrangement shown in the figures includes:
a short tubular catheter (1) equipped with a proximal base (2);
a needle (3) which has a skin-puncture end (3a) and which is equipped with a proximal base (4);
a cage (5) composed of a rear part (6) and a front part (7);
a mobile retention device (8).

The retention device is a moulded body which has two walls (9, 10) set at right angles. One of the walls (9) ends in a dog (11), and the other wall (10) is drilled with a hole (12) for the passage of the needle.

The rear part (6) of the cage forms a chamber (13) for the passage of the needle through the cage and forms the entrance (14) of this chamber, while the front part (7) of the cage forms a transverse wall (15) to close off the chamber, this wall being drilled with a hole (16) which constitutes the exit from the chamber. In front of this wall, the front part (7) forms a nose (17) which is designed to fit into in the catheter base.

The rear part (6) of the cage is shaped so that the base of the needle slots into the rear part for the puncture operation (FIGS. 2 and 3).

The transverse wall (15) of the front part has lateral nipples (18) designed to click into holes (19) in the wall of the chamber of the rear part, so as to secure the two parts.

This transverse wall also forms slides (20) for sliding the wall (10) of the retention device.

Finally, this transverse wall is equipped with a nipple (21) which is designed to fit into in an oblong aperture (22) in the sliding wall (10) in order to limit the lifting of the mobile device.

Close to its entrance, the base (2) of the short catheter has a rim (23), such as a continuous or discontinuous external collar, to stop the dog (11) of the guillotine, and this dog has a ramp (24) which is designed to slide progressively on the collar when the cage is drawn in the proximal direction (FIGS. 6 to 8), which causes the lifting of the guillotine until this lifting is stopped by the nipple (21), as shown in particular in FIG. 8. The positions of the nipple (21) and of the aperture (22) are adjusted so that stopping does not occur before the dog has risen above the collar (FIG. 7).

Resources are also provided to prevent the guillotine from returning to the down position after it has reached the up position.

In the example shown, these resources take the form of flexible and elastic legs (8) formed on the sides of the sliding wall (10) of the mobile device, and which are constrained by rims (25) on the transverse wall (15) of the front part (7) of the cage until these legs have arrived at the top of the rims during the lifting of the guillotine (FIGS. 11 and 12), a position at which the legs move outwards and rest on the rims.

Preferably, resources are also provided, in a manner which is known of itself, to prevent the puncture end of the needle from leaving the cage via the proximal entrance of the chamber.

It is also proposed that the cage be connected to the base of the needle by a deployable link so that, in the deployed state, this link has a length that is less than the length of the needle. (WO 94100172, U.S. Pat. No. 5,176,655, U.S. Pat. No. 6,234,999, U.S. Pat. No. 6,001,080).

It is also proposed to limit the proximal entrance of the cage to a simple hole for the passage of the needle, and to equip the needle with a local bulge so that sliding the needle in the proximal direction is halted by jamming of this bulge against the periphery of the hole.

This present invention is not based on any particular choice of such an arrangement and, purely as an example, a description is provided of an arrangement composed of a fixed transverse wall (26) in which the proximal entrance (14) of the cage is created, together with a local bulge (27) in the cross section of the needle. This bulge has been shown on some figures only, and in a somewhat exaggerated manner. It goes without saying that this bulge is designed to as not to prevent the needle from sliding in the cannula.

The invention is not limited only to the implementations which have been described.

1 An arrangement for the insertion, into a vein, of a cannula composed of a short catheter (1) with a proximal base (2), where this arrangement includes a needle (3) which has a puncture end (3a) and a cage (5) which extends the base of the cannula in the proximal direction, where this chamber forms a chamber (13) through which the needle slides from a proximal entrance (14) to a distal exit (16), and is equipped with a trap to hold the puncture end of the needle in the chamber when the needle is withdrawn from the catheter base, with the cage and the base being equipped with retention resources that combine so as to perform temporary retention of the cage on the base before the puncture end of the needle is trapped in the chamber of the cage, with the said retention resources including an external rim (23) formed on the base, and an external dog (11) provided on the cage to be held by this rim, characterised in that the dog is formed on a mobile device (9, 10) which includes a wall (10) traversed by a hole (12) for the passage of the needle so that the passage of the needle in the hole holds the dog in a retention position, the said wall (10) being mounted to slide in the chamber of the cage in a direction that is transverse to the needle, between a down held position and an up released position, so that when the needle is withdrawn into the cage until it has moved to the rear of the said hole, the wall (10) which is not traversed by the needle can slide to its up position, lifting the said dog and allowing the cage to separate from the catheter base.

2. An arrangement according to claim 1, in which the said dog (11) is formed at the end of a wall (9) of the retention device which is at right angles to the sliding wall (10).

3. An arrangement according to claim 1 in which the said rim (23) and/or the said dog (11) has a ramp (24) that a reverse movement of the cage in the proximal direction causes the dog to lift when the needle (3) no longer traverses the said hole (12).

4. An arrangement according to claim 1, and which includes resources (21, 22) in order to limit the 10 lifting of the sliding wall in the up position.

5. An arrangement according to claim 1, and which includes resources (8, 8) to prevent the mobile device returning to the down position after it has reached the up position.

6. An arrangement according to claim 1, in which the cage (5) is formed of a rear part (6) which constitutes the chamber (13) and the entrance (14) of the chamber, and a front part (7) which includes a transverse wall (15) which closes off the said chamber and which includes the exit (16) from the chamber, this transverse wall (15) forming a slide for the said sliding wall (10) of the said retention device, these two parts (6, 7) being held together by a click on action of nipples (18) provided on the sides of the transverse wall of the front part, in holes (19) formed in the wall of the chamber of the rear part, or vice versa.

7. An arrangement according to claim 6 in which, ahead of the said transverse wall (15), the front part (7) of the cage constitutes a nose (17) designed to fit into in the catheter base.

8. An arrangement according to claim 6, in which the said transverse wall (15) of the front part (7) of the cage includes a nipple (21) which slides in an oblong aperture (22) of the sliding wall of the retention device in order to limit the lifting of the mobile device.

9. An arrangement according to claim 6, in which the said sliding wall (10) of the retention device has flexible and elastic lateral legs (8, 8) which are constrained by rims (25) formed in the transverse wall (15) of the front part (7) of the cage for as long as the said device has not yet reached the up position, and which deploy when the device reaches the up position, to rest on these rims and so prevent the device from returning to the down position.

10. An arrangement according to claim 6 in which the rear part of the cage is shaped so that the base of the needle slots into this part for the puncture operation.

11. An arrangement according to claim 1, and which includes resources (14, 27) to prevent the point of the needle exiting via the entrance of the chamber.

12. An arrangement according to claim 2, in which the said rim (23) and/or the said dog (11) has a ramp (24) so that a reverse movement of the cage in the proximal direction causes the dog to lift when the needle (3) no longer traverses the said hole (12).

13. An arrangement according to claim 2, and which includes resources (21, 22) in order to limit the 10 lifting of the sliding wall in the up position.

14. An arrangement according to claim 2, and which includes resources (8, 8) to prevent the mobile device returning to the down position after it has reached the up position.

15. An arrangement according to claim 2, in which the cage (5) is formed of a rear part (6) which constitutes the chamber (13) and the entrance (14) of the chamber, and a front part (7) which includes a transverse wall (15) which closes off the said chamber and which includes the exit (16) from the chamber, this transverse wall (15) forming a slide for the said sliding wall (10) of the said retention device, these two parts (6, 7) being held together by a click on action of nipples (18) provided on the sides of the transverse wall of the front part, in holes (19) formed in the wall of the chamber of the rear part, or vice versa.

16. An arrangement according to claim 15 in which, ahead of the said transverse wall (15), the front part (7) of the cage constitutes a nose (17) designed to fit into in the catheter base.

17. An arrangement according to claim 15, in which the said transverse wall (15) of the front part (7) of the cage includes a nipple (21) which slides in an oblong aperture (22) of the sliding wall of the retention device in order to limit the lifting of the mobile device.

18. An arrangement according to claim 15, in which the said sliding wall (10) of the retention device has flexible and elastic lateral legs (8, 8) which are constrained by rims (25) formed in the transverse wall (15) of the front part (7) of the cage for as long as the said device has not yet reached the up position, and which deploy when the device reaches the up position, to rest on these rims and so prevent the device from returning to the down position.

19. An arrangement according to claim 15 in which the rear part of the cage is shaped so that the base of the needle slots into this part for the puncture operation.

20. An arrangement according to claim 2, and which includes resources (14, 27) to prevent the point of the needle exiting via the entrance of the chamber.
